(19) United States
(12) Patent Application Publication
(10) Pub. No.: US 2011/0059438 A1
(43) Pub. Date: Mar. 10, 2011

(54) Method of Preparing DNA Fragments and Applications Thereof

(75) Inventors: Anne-Gaelle Brachet, Nantoin (FR); Philippe Rizo, La Tronche (FR)

(73) Assignee: COMMISSSARIAT A L'ENERGIE ATOMIQUE, Paris (FR)

(21) Appl. No.: 12/458,610

(22) Filed: Jul. 16, 2009

Related U.S. Application Data
(63) Continuation of application No. 10/549,137, filed on Aug. 9, 2006, now abandoned.

(30) Foreign Application Priority Data
Mar. 18, 2003 (FR) 0303294
Mar. 18, 2004 (FR) PCT/FR04/00671

(51) Int. Cl. C12Q 1/68; C12P 19/34; C07H 21/04

(52) U.S. Cl. 435/6; 435/91.1; 536/23.1; 536/24.33

(57) ABSTRACT

A method of preparing DNA fragments, which entails at least the following steps of:
a) preparing double-stranded DNA fragments from a sample of nucleic acids to be analyzed,
b) ligating the ends of said DNA fragments to a double-stranded oligonucleotide adaptor (adaptor AA′) comprising the recognition site for a restriction enzyme, the cleavage site of which is located downstream of said recognition site,
c) amplifying the fragments linked to said adaptor, using a pair of suitable primers, at least one being optionally labeled at its 5′ end, and
d) cleaving said DNA fragments close to one of their ends, using said restriction enzyme, so as to generate short fragments.

### FIELD OF THE INVENTION

The invention relates to a method of preparing DNA fragments and to applications thereof, in particular for the hybridization of nucleic acids.

### DESCRIPTION OF THE BACKGROUND

Many techniques based on the principle of hybridization of nucleic acid molecules having complementary sequences are used in extremely varied fields of biology, in particular for detecting the presence of nucleic acids (mRNA, DNA.) using samples to be analyzed, identifying possible variations in their sequence or else determining this sequence. By way of non-limiting example, mention may be made of: genome analysis, genetic mapping genotyping and the identification of species, of varieties or of individuals (animal, plant, microorganism) by investigating genetic fingerprints (DNA fingerprinting), the detection of a polymorphism (SNP or single nucleotide polymorphism), the search for mutations or genes associated with phenotypic characteristics, and minisequencing, and also transcriptome analysis, in particular the establishment of gene expression profiles.

In general, the hybridization is carried out on samples consisting of double-stranded DNA (genomic DNA extract or cDNA synthesized from an RNA extract). The double-stranded DNA is fragmented using one or more restriction enzymes, the fragments of approximately 200 to 400 bp are purified, covalently linked—by hybridization (sticky ends) and then ligation using ligase (blunt or sticky ends)—to double-stranded oligonucleotides (adaptors), the end of which corresponds to the sequence of the restriction site(s) of said enzymes, and the fragments are then amplified by polymerase chain reactions (PCRs) using oligonucleotide primers which include the above restriction site(s) and at least one of which is labeled at its 5′ end, so as to obtain a sufficient amount of labeled targets for hybridization with the probe.

A set number of methods using one or more adaptors have been described: application EP 0 534 858 in the name of Keygene, PCT international application WO 02/34939, the method described in the article in the names of K. Kato et al. (N. A. R., 1995, 23, 3685-3690), American application US 2002/072055, PCT International application WO 94/01582 and American application US 2003/008292.

The PCR products thus obtained constitute the targets which are hybridized with one or more probes immobilized on an appropriate support (plastic, nylon membrane, glass, gels, silicon, etc.), each probe consisting of a single-stranded nucleic acid molecule, the sequence of which is complementary to all or part of that of the target. Miniaturized supports to which many probes are attached (DNA chips) thus make it possible to simultaneously visualize hundreds of reactions consisting of hybridization of (labeled) target fragments with specific probes.

Other methods, that do not use a PCR step, have also been described: PCT international application WO 00/15368 and PCT international application WO 98/10095.

None of these methods makes it possible to improve the sensitivity, the specificity, the simplicity and the rapidity of the nucleic acid hybridization methods.

Many applications, in particular those that involve the distinction of one base between the sequence of the target and of the probe (SNP detection), require the use of short probes. In this case, the hybridization of PCR products, i.e. of targets of several hundred base pairs, with probes of 10 to 20 bases is often of poor quality (low signals, false negatives and false positives) for the following reasons:
the presence of secondary structures in the target decreases the efficiency of hybridization of the probe, due to the decrease in accessibility to the target and to the fact that it is impossible to optimize the hybridization conditions because of the presence of a large number of fragments, having different structures, to be hybridized with the same probe, and
non-specific hybridization or cross hybridization reactions with “non-target”, sequences having similarities with the target sequence lead to false positives that reduce the ability to detect small amounts of specific sequences and the ability to discriminate these sequences, due to the increase in background noise.

Thus, various improvements have been proposed in order to increase the sensitivity (weak signals, false negatives) and the specificity (false positives) of these techniques:
increase in the hybridization time (of the order of 12 h to 18 h; Dai et al., NAR, 2002, 30 (13), e86; Ramakrishnan et al., NAR, 2002, 30, 1-12; Rodriguez et al., Molecular Biotechnology, 1999, 11, 1 to 12; Kane et al., NAR, 2000, 28, 4552-4551); this approach, which makes it possible to obtain a good hybridization signal specific for the sequence to be detected, is incompatible with the current objectives of high-throughput analysis involving the rapid processing of a large number of samples (miniaturization, running experiments in parallel, etc.),
hybridization of the probe with a single-stranded PCR product obtained by means of an asymmetric PCR reaction (Guo et al., Genome Research, 2002, 12, 445-457); this solution, which makes it possible to increase the hybridization signal by a factor of 4 to 5, involves additional steps of purification of the double-stranded PCR product and of amplification of a labeled single-stranded PCR fragment,
optimization of the length and of the composition of the sequence of the probe, using appropriate programs; this solution, which makes it possible to improve the quality of the hybridization by limiting the number of secondary structures and by making the probe hybridization temperatures homogeneous, does not solve the problems of cross reactions related to the size and to the structure of the targets,
use of auxiliary oligonucleotides (Rodriguez et al., mentioned above), consisting of pre-hybridization of the target with random and varied short oligonucleotide sequences before the step consisting of hybridization with the probe, with the aim of cleaving the secondary structures of the target in the region to be analyzed and of limiting the drop in hybridization yield, due to the presence of redundant sequences in the target; this strategy is expensive and is not efficient, since the oligonucleotides that are added in the end compete with the probe and decrease the hybridization signal.

It emerges from the above that there exists a real need for providing methods of nucleic acid hybridization that are more suited to practical needs, in particular in that they are rapid, sensitive, specific and simple to carry out. Such methods that thus make it possible to simultaneously analyze a large number of samples on supports of the DNA chip type, whatever the technique used, would therefore be entirely suitable for all the above-mentioned applications, in the genomics and proteomics field.

It is for this reason that the inventors have developed a method of preparing DNA fragments that advantageously makes it possible to obtain short DNA fragments and, consequently, to obtain rapid, efficient and specific hybridization of nucleic acid molecules (DNA, RNA); said method is useful both for preparing target DNAs capable of hybridizing with nucleotide probes, and in particular with oligonucleotide probes, and for preparing DNA probes, in particular DNA chips, capable of hybridizing with target nucleic acids (DNA, RNA).

### SUMMARY OF THE INVENTION

A subject of the present invention is thus a method of preparing DNA fragments, characterized in that it comprises at least the following steps:
a) preparing double-stranded DNA fragments from a sample of nucleic acids to be analyzed,
b) ligating the ends of said DNA fragments to a double-stranded oligonucleotide adaptor (adaptor AA′) comprising the recognition site for a restriction enzyme, the cleavage site of which is located downstream of said recognition site,
c) amplifying said fragments linked to said adaptor, using a pair of suitable primers, at least one being optionally labeled at its 5′ end, and
d) cleaving said DNA fragments close to one of their ends, using said restriction enzyme, so as to generate short fragments,

For the purpose of the present invention, the term “short fragment” is intended to mean a fragment of less than 100 bases or 100 base pairs, preferably of approximately 20 to 50 bases or base pairs.

The method of preparing DNA fragments according to the invention advantageously makes it possible to obtain short fragments, i.e. of a length equivalent to that of the oligonucleotide probes; the use of such short fragments as targets or probes in hybridization techniques has the following advantages compared with the hybridization techniques of the prior art:

Sensitivity and Specificity

The sensitivity and the specificity of the hybridication are increased due to:
the decrease in cross hybridization reactions and false positives, through elimination of the “non-target sequences”,
the increase in hybridization signal through the decrease in secondary structures of the DNA,
the harmonization of the hybridization conditions (temperature),
the purity of the DNA (elimination of the enzyme, buffers and long DNA fragments that remain).

Simplicity

The preparation of DNA fragments (target or probe) comprises steps that are simple to carry out (enzymatic digestion, ligation and PCR amplification). In addition, optimization of the DNA (target or probe) makes it possible to obtain a hybridization of good quality (no false positives, little background noise, etc.) and therefore to minimize the number of controls that are necessary and, consequently, to reduce the complexity of the chip.

Rapidity

The hybridization time is significantly reduced and is less than 1 h (approximately 15 to 20 min), instead of 12 h to 18 h in the techniques of the prior art.

Relatively Low Cost

The method of preparing DNA according to the invention is relatively inexpensive, compared with the use of auxiliary oligonucleotides.

In addition, the reduction in complexity of the chip makes it possible to reduce the cost of the latter.

Because of these various advantages, the method preparing DNA fragments according to the invention is particularly well suited to:
the rapid analysis of a large number of target DNA samples on DNA chips, whatever the hybridization technique used, and consequently whatever the applications envisioned (minisequencing, genotyping, search for polymorphism by SNP, establishment of gene expression profiles),
the preparation of probes of small and controlled size, from genomic DNA or RNA, in particular for producing DNA chips on which said probes are immobilized.

In accordance with the method of the invention, steps a) and b) are carried out successively or simultaneously.

In accordance with the method of the invention, the double-stranded DNA fragments of step a) are obtained by conventional techniques that are known in themselves. For example, the genomic DNA extracted from the sample to be analyzed is fragmented randomly using one or more endonuclease(s) (restriction enzyme) selected according to its (their) frequency of cleavage of the DNA to be analyzed, so as to obtain fragments of less than 1000 bp, of the order of 200 to 400 bp. The RNA (mRNA, genomic RNA from a microorganism, etc.) is extracted from the sample to be analyzed, converted to double-stranded cDNA by reverse transcription, and then fragmented in a similar manner to the genomic DNA. Among the endonucleases that can be used to cleave mammalian DNA, mention may be made of restriction enzymes for which the recognition site and the DNA cleavage site are combined, f or instance the type II restriction enzymes, such as, without implied limitation: EcoR I, Dra I, Ssp I, Sac I, BamH I, BbvC I, Hind III, Sph I, Xba I and Apa I.

In accordance with the method of the invention, the adaptor of step b) is an oligonucleotide of at least 6 bp, formed from two complementary strands (A and A′, FIG. 2) comprising the recognition site for a restriction enzyme (zone 2), the cleavage site of which is located downstream of the recognition site. Among these restriction enzymes, mention may be made of the restriction enzymes of type IIS or F, such as, without implied limitation: Bpm I, Bsg I and BpuE I, which cleave 16 nucleotides downstream of their recognition site, and Eci I, BsmF I, Fok I, Mme I and Mbo II, which cleave, respectively, 11, 10, 9, 20 and 8 nucleotides downstream of their recognition site. Preferably, said adaptor is formed from the combination of two complementary oligonucleotides, the sequence of which is respectively that of the strands A and A′ as defined above. Said adaptor is linked to the ends of said DNA fragment by any suitable means, known in itself, in particular using a DNA ligase, such as T4 ligase.

In accordance with the method of the invention, the amplification in step c) is carried out using a primer comprising the sequence of the oligonucleotide A of the adaptor. For example, the sequence of the primer is either that of the oligonucleotide A or that of the latter to which are added, in the 3′ position, the bases corresponding to the overhanging sequence of the ends of the fragment from step a), generated by the endonuclease used in step a), as defined above (primer B, FIG. 3). In accordance with the method of the invention, the cleavage at the end of the double-stranded DNA fragment in step d) makes it possible to obtain short DNA fragments that may contain the sequence to be detected (informative sequence) by hybridization with a specific nucleotide probe, in particular an oligonucleotide complementary to said informative sequence.

According to an advantageous embodiment of the method according to the invention, steps a) and b) are carried out simultaneously.

According to another advantageous embodiment of the method according to the invention, it comprises an additional step consisting in purifying the fragments of less than 1000 bp, prior to the ligation step b). Said purification is carried out by any suitable means known in itself, in particular by separation of the digestion products obtained in a) by agarose gel electrophoresis, visualization of the bands corresponding to the various fragments obtained, removal of the gel band (s) corresponding to the fragments of less than 1000 bp, and extraction of said double-stranded DNA fragments according to conventional techniques.

According to yet another advantageous embodiment of the method according to, the invention, said adaptor of at least 6 bp (step b) comprises, upstream of the recognition site (zone 2), a zone 3 of at least 6 base pairs; such a zone makes it possible to improve the hybridization by extension of the adaptor (FIG. 2). The sequence of zone 3 is selected by any suitable means known in itself, in particular using programs for predicting suitable sequences that make it possible to optimize the length, the structure and the composition of the oligonucleotides (GC percentage, absence of secondary structures and/or of self-pairing, etc.).

According to yet another advantageous embodiment of the method according to the invention, said adaptor (step b) comprises on one of the strands (A or A′), downstream of the recognition site (zone 2), a zone 1 complementary to the overhanging sequence of the ends of the fragment of step a), generated by the endonuclease used in step a), as defined above (FIG. 2). Preferably, said adaptor comprises at least one base located between zone 1 and zone 2 that is different from that which, in said restriction site, is immediately adjacent to the above complementary sequencer this base makes it possible not to reconstitute said restriction site after the ligation of the adaptor in step b) and therefore to prevent cleavage of the adaptor linked to the end of said double-stranded DNA fragment.

According to yet another advantageous embodiment of the method according to the invention, said adaptor (step b) comprises a phosphate residue covalently linked to the 5′ end of the strand A′; this phosphate residue enables an enzyme (for example, a DNA ligase such as T4 ligase) to link said adaptor to the 3′-OH ends of the double-stranded DNA fragment, via a phosphodiester bond. According to yet another advantageous embodiment of the method according to the invention, one of the primers (step c) is linked at its 5′ end to a suitable label for detecting nucleic acid hybrids (DNA-DNA, DNA-RNA), for example a fluorophore.

According to yet another advantageous embodiment of the method according to the invention, said primers (step c) contain, at their 3′ end, several bases specific for an informative sequence or informative sequences to be detected, so as to amplify only some of the fragments (differential amplification), in particular in order to prevent saturation of the chip with too great a number of target DNA fragments.

According to yet another advantageous embodiment of the method according to the invention, one of the strands of the product amplified in step c) is protected at its 5′ end with a suitable label; it is thus possible to eliminate the complementary strand by the action of a phosphatase and then of a 5′ exonuclease. The labeled strand is not destroyed by the enzyme, since the label prevents the exonuclease from progressing along the strand and therefore digesting it.

According to yet another advantageous embodiment of the method according to the invention, it comprises an additional step e) consisting in obtaining, by any suitable means, single-stranded fragments from the short fragments obtained in step d).

According to yet another advantageous embodiment of the method according to the invention, it comprises an additional step e′), consisting in purifying, by any suitable means, the short fragments obtained in step d), or a step f) consisting in purifying the single-stranded fragments obtained in step e). In accordance with the method of the invention, single-stranded DNA fragments are obtained by any suitable means known in itself, for example through the action of an alkaline phosphatase and then of 5′ exonuclease.

In accordance with the method of the invention, the short, optionally single-stranded, fragments are purified by any suitable means known in itself, for example: exclusion chromatography, filtration, precipitation with mixtures of ethanol and of ammonium acetate or sodium acetate.

A subject of the present invention is also a short single-stranded DNA fragment that can be obtained by means of the method as defined above, characterized in that it is less than 100 bases or base pairs long and in that it comprises at least one informative sequence bordered at its 5′ and 3′ ends, respectively, by the recognition site and the cleavage site for a restriction enzyme that cleaves at a distance from its recognition site.

In accordance with the invention, the informative sequence or target sequence corresponds to the sequence of a sample of nucleic acids to be analyzed, which is detected specifically by the probe used for the hybridization; said informative sequence represents, for example, a genetic marker useful for detecting a species, a variety or an individual (animal, plant, microorganism) or an area of polymorphism, or else a cDNA marker specific for a protein, useful for studying transcriptomes and establishing gene expression profiles.

In accordance with the invention, said short single-stranded DNA fragment may also comprise, upstream and/or downstream of the recognition site for said restriction enzyme, the sequences corresponding to zone 1 and to zone 3, as defined above.

According to an advantageous embodiment of the invention, said short single-stranded DNA fragment is labeled at its 5′ end with a suitable label for detecting DNA-DNA hybrids, for example a fluorophore.

According to another advantageous embodiment of the invention, said short single-stranded DNA fragment is immobilized on a suitable support. The supports on which nucleic acids can be immobilized are known in themselves; by way of non-limiting example, mention may be made of those which are made of the following materials: plastic, nylon, glass, acrylamide, etc.) and silicon.

Preferably, said DNA fragment is immobilized on a miniaturized support of the DNA chip.

A subject of the present invention is also a DNA chip, characterized in that it comprises a short single-stranded DNA fragment as defined above.

A subject of the present invention is also a method of hybridizing nucleic acids, characterized in that it uses:
a probe or a target consisting of a short single-stranded DNA fragment as defined above, and/or
a probe consisting of a short double-stranded DNA fragment formed from the association of the short single-stranded DNA fragment as defined above and of the sequence complementary thereto.

A subject of the present invention is also a kit for carrying out a method of hybridization, characterized in that it comprises at least one DNA fragment (target or probe) as defined above and a nucleic acid molecule complementary to said DNA fragment, in particular an oligonucleotide probe.

A subject of the present invention is also the use of an adaptor as defined above for preparing short single-stranded DNA fragments as defined above.

A subject of the present invention is also the use of a primer as defined above for preparing short single-stranded DNA fragments as defined above.

A subject of the present invention is also an adaptor formed from a double-stranded oligonucleotide (AA′) of at least 10 bp comprising, from 5′ to 3′ (FIG. 2):
a zone 3 of at least 6 bp, as defined above,
a zone 2 comprising the recognition site for a restriction enzyme, the cleavage site of which is located downstream of the recognition site,
a zone 1 complementary to the overhanging sequence of the ends of the fragment of step a) of the method as defined above, generated by the endonuclease used in step a) as defined above,
at least one base located between zone 1 and zone 2 that is different from that which, in said restriction site for said endonuclease used in step a), is immediately adjacent to the complementary sequence of said zone 1, and
a phosphate residue covalently linked to the 5′ end of the strand A′.

A subject of the present invention is also a primer, characterized in that it comprises the sequence of the oligonucleotide A of the adaptor. Preferably, the sequence of said primer is selected from the group consisting of: the sequence of the oligonucleotide A, and the sequence of the latter, to which are added, in the 3′ position, bases corresponding to the overhanging sequence of the ends of the fragment of step a), generated by the endonuclease used in step a), as defined above (primer B, FIG. 3).

A subject of the present invention is also a kit for carrying out the method as defined above, characterized in that it comprises at least one adaptor and a pair of primers as are defined in the method above.

### BRIEF DESCRIPTION OF THE DRAWINGS

Besides the above arrangements, the invention also comprises other arrangements that will emerge from the following description, which refers to examples of implementation of the method of preparing DNA fragments according to the invention and of its use for hybridizing nucleic acids, in particular to oligonucleotide probes, and also refers to the attached drawings in which:

FIG. 1 illustrates the principle of the method of preparing DNA fragments (target or probe) according to the invention;

FIG. 2 illustrates the general structure of the adaptor (AA′);

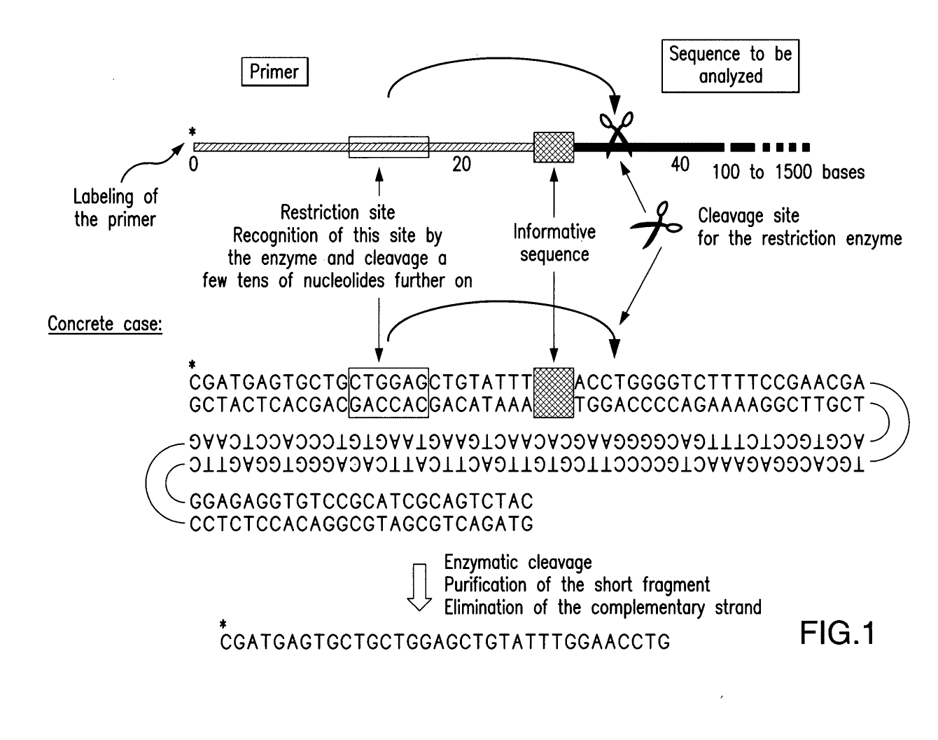

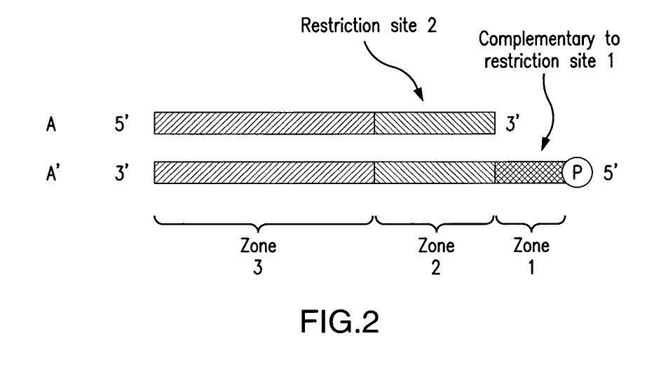

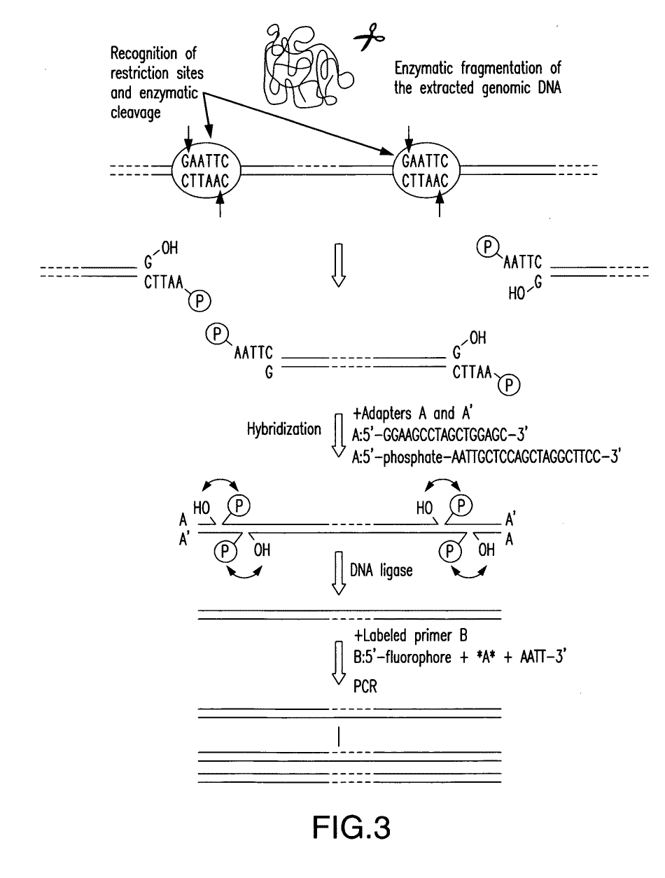

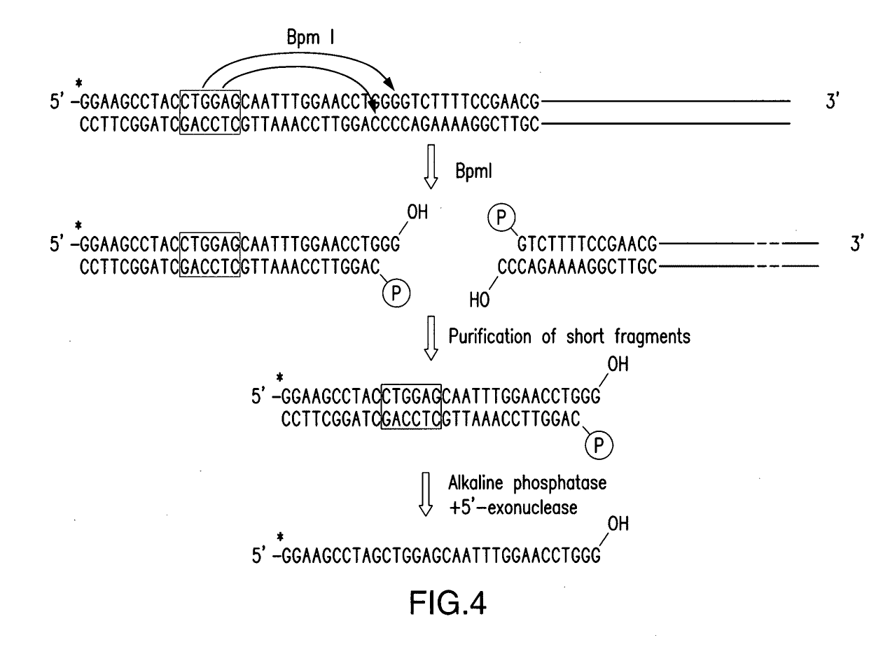

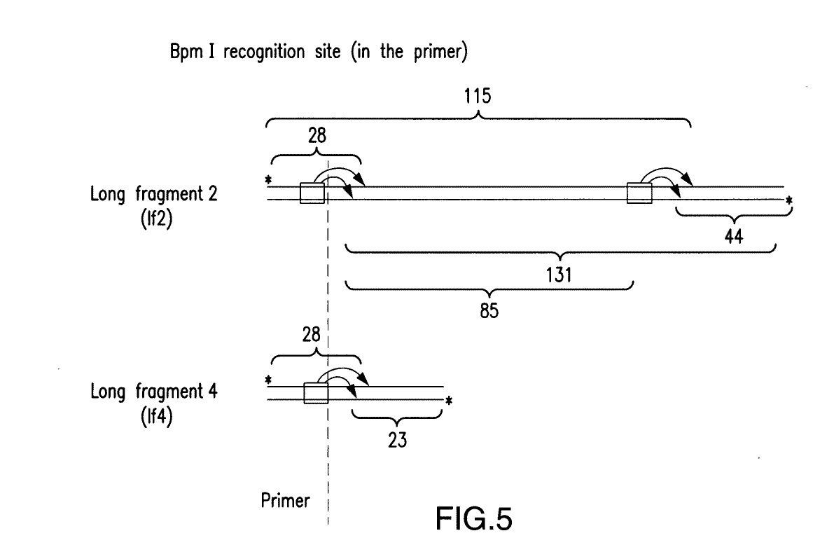

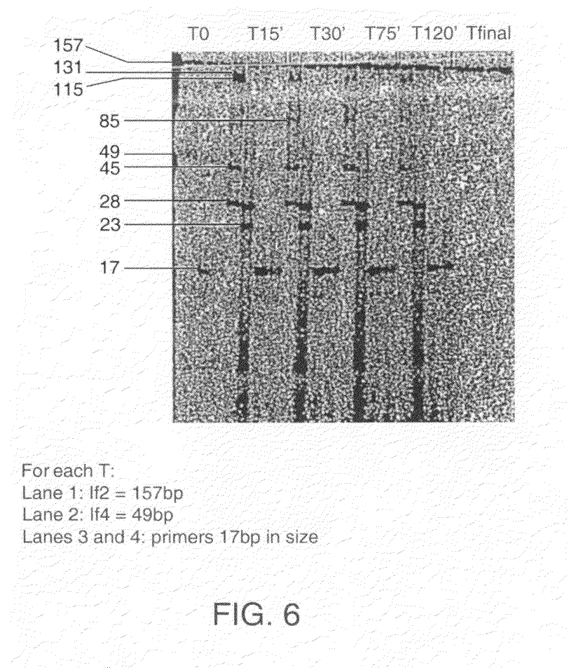

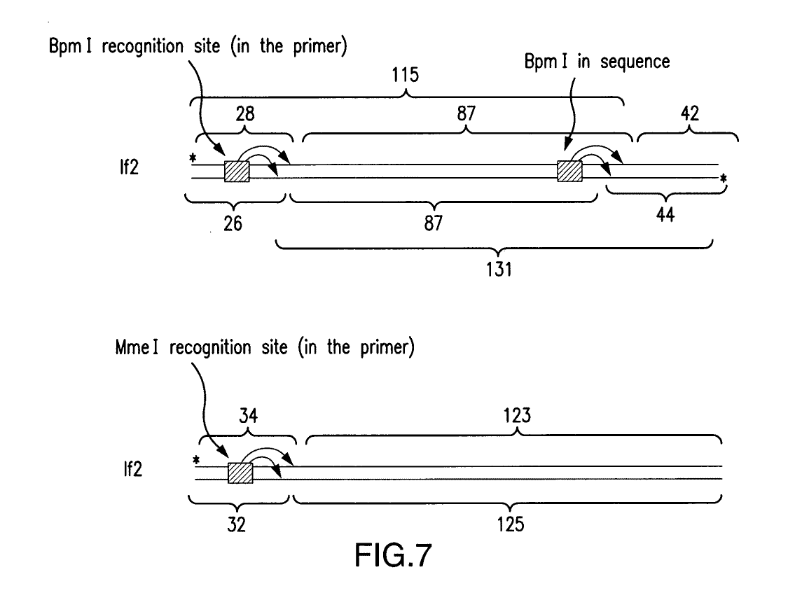

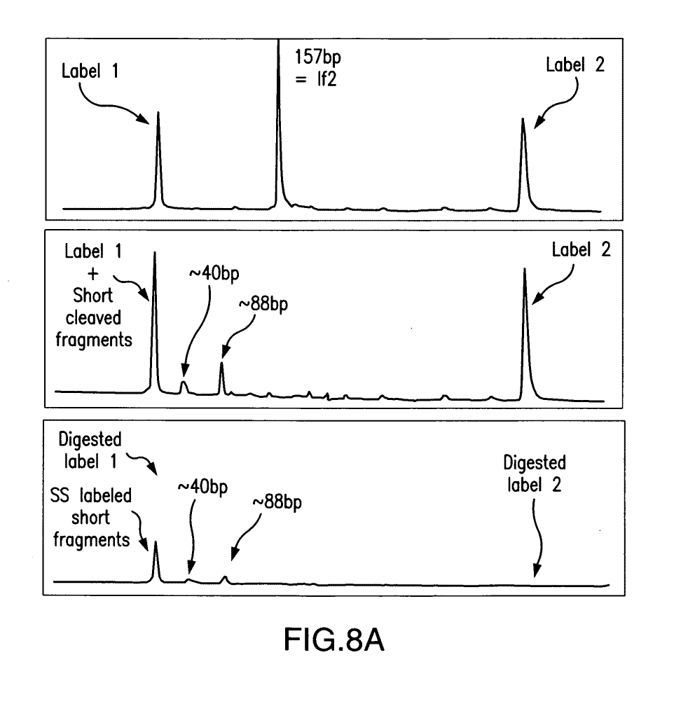

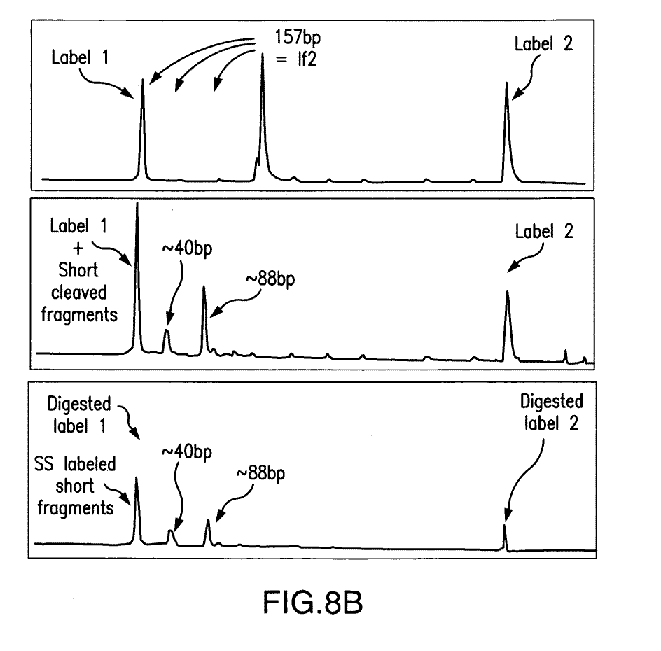

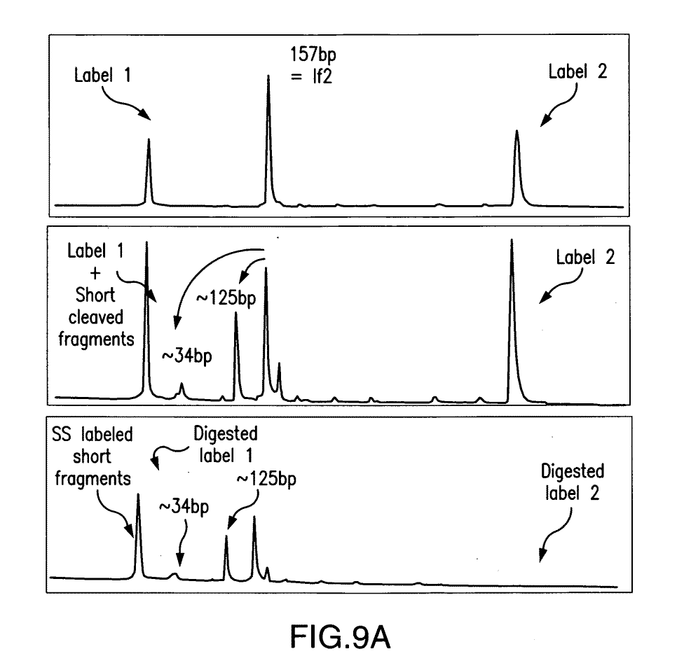

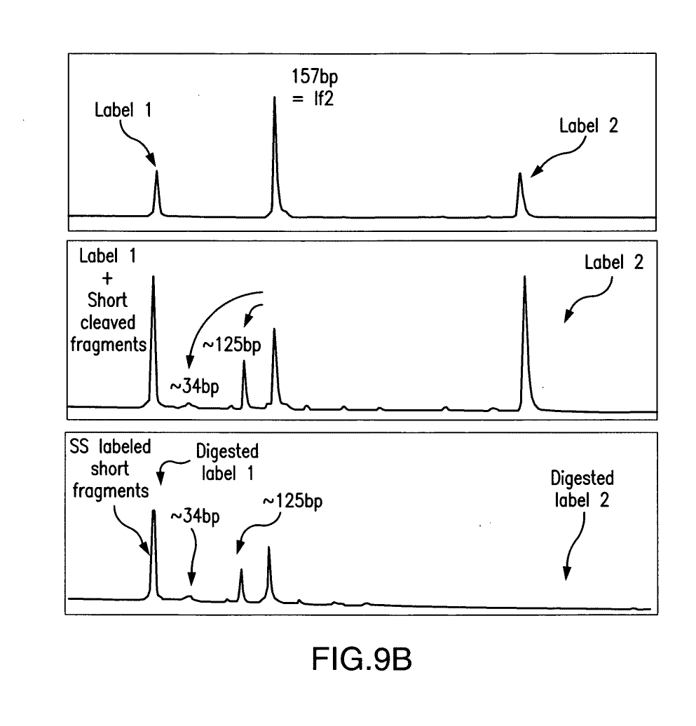

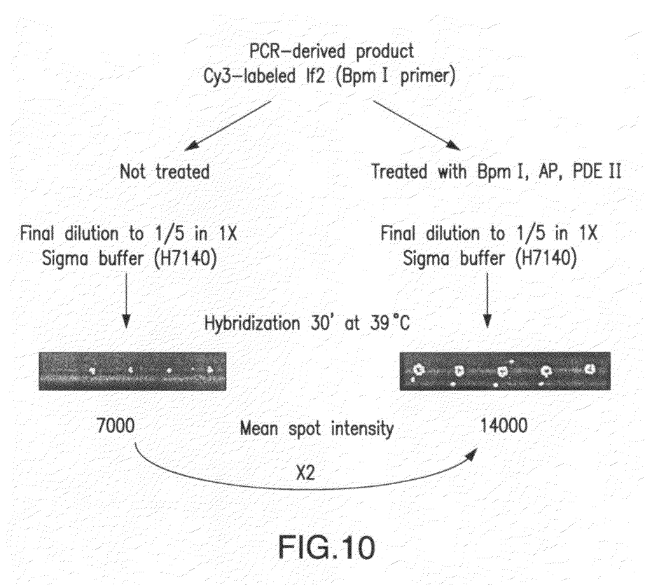

FIG. 3 illustrates an example of steps a) to c) of the method of preparing DNA fragments according to the invention:
step a): the double-stranded DNA fragments are generated by cleavage with EcoR I which recognizes the site GAATTC,
step b): the adaptor AA′ ( 16/20 bp) comprises, respectively, from 5′ to 3′: a sequence of 10 base pairs (zone 3: sequence 5′ GGAAGCCTAG 3′ on the strand A), the recognition site for the Bpm I enzyme (zone 2: sequence 5′ CTGGAG 3′ on the strand A), and also the sequence complementary to the EcoR I site and an additional base pair, and also a phosphate residue at the 5′ end of the strand A′ (zone 1: sequence 5′, phosphate-AATTG on the strand A′). The DNA ligase makes it possible to link the adaptor to the sticky ends of the EcoR I fragments via phosphodiester bonds, and
step c): the fragments linked to the adaptor are amplified by PCR using primer B (21 bases), primer B being labeled at its 5′ end with a fluorophore;

FIG. 4 illustrates an example of steps d) and e) of the method of preparing target DNAs according to the invention: the labeled fragments obtained in step c) are cleaved at their 5′ end, using the Bpm I enzyme which cleaves 16 nucleotides downstream of the recognition site (14 nucleotides downstream on the complementary strand), so as to generate short fragments (32/30 Up) which are purified, and then the nonlabeled complementary strand is eliminated by digestion, successively, with an alkaline phosphatase and a 5′ exonuclease. The labeled DNA fragments thus obtained are 32 bases in length, which bases comprise 12 bases of informative sequence, specific for the nucleic acids to be analyzed;

FIG. 5 represents the restriction map for the long fragments lf2 and lf4 with Bpm I;

FIG. 6 represents the polyacrylamide (20%) gel profile of the radiolabeled short fragments obtained after cleavage of the long fragments lf2 and lf4 with Bpm I. For each incubation time (T) with the Bpm I restriction enzyme (0, 15, 30, 75, 120 minutes and final T), lane 1 corresponds to lf2 (157 bp), lane 2 corresponds to lf4 (49 bp) and lanes 3 and 4 correspond to the primers (17 bp);

FIG. 7 represents the restriction map for the long fragment lf2 with Bpm I and Mme I;

FIGS. 8A and 8B represent the profile of the fragments obtained after cleavage, with Bpm I, of the long fragment lf2 labeled in the 5′ position with Cy3 (central panel in A) or fam (fluorescein acetoxymethy ester) (central panel in B). The upper panel in A and B corresponds to the profile of the fragment lf2 not cleaved with Bpm I. The lower panel in A and B corresponds to the profile of the fragment lf2 cleaved with Bpm I and digested with alkaline phosphatase and the 5′ exonuclease PDE II;

FIGS. 9A and 9B represent the profile of the fragments obtained after cleavage, with Mme I, of the long fragment lf2 labeled in the 5′ position with Cy3 (central panel in A) or fam (central panel in B). The upper panel in A and B corresponds to the profile of the fragment lf2 not cleaved with Mne I. The lower panel in A and B corresponds to the profile of the fragment lf2 cleaved with Mme I and digested with alkaline phosphatase and the 5′ exonuclease PDE II;

FIG. 10 illustrates the analysis of the intensity of the hybridization signal for a short double-stranded or single-stranded target, compared with a long double-stranded target.

### DETAILED DESCRIPTION OF THE PREFERRED EMBODIMENT

### Example 1

### Preparation of DNA Fragments (Target or Probe) According to the Method of the Invention

The preparation of the nucleic acids, the enzymatic digestions, the ligations, the PCR amplifications and the purification of the fragments thus obtained were carried out using conventional techniques according to standard protocols, such as those described in Current Protocols in Molecular Biology (Frederick M. Ausubel, 2000, Wiley and Son Inc., Library of Congress, USA).

More specifically, DNA fragments were prepared in the following way:

The genomic DNA was extracted from bovine blood (Bos taurus) using the PAXgene Blood DNA kit (reference 761133, Qiagen), according to the manufacturer's instructions.

The following adaptors and primers were synthesized by MWG Biotech:

adaptor
(SEQ ID NO: 1)
strand A: 5′-GGAAGCCTAGCTGGAGC-3′
(SEQ ID NO: 2)
strand A′: 5′-P-AATTGCTCCAGCTAGGCTTCC-3′
primer
(SEQ ID NO: 3)
B: 5′-Cy-GGAAGCCTAGCTGGAGCAATT-3′.

The purified genomic DNA (5 μg) and the adaptor (5 μg) were incubated at 37° C. for 3 h in 40 μl of 50 mM Tris-HCl buffer, pH 7.5, 10 mM MgCl₂, 50 mM NaCl, 10 mM DTT, 1 mM ATP and 1 mg BSA, containing 50 IU of EcoR 1 and 2 IU of T4 DNA ligase. The DNA fragments linked at their ends to the adaptor AN thus obtained were amplified by PCR using primer B in a reaction volume of 50 μl containing: 1 ng of DNA fragments, 150 ng of the primer and 2 IU of AmpliTaq Gold® (Perkin Elmer) in a 15 mM Tris-HCl buffer, pH 8.0, 10 mM KCl, 5 mM MgCl₂ and 200 μM dNTPs. The amplification was carried out in a thermocycler, for 35 cycles comprising: a denaturation step at 94° C. for 30 s, followed by a hybridization step at 60° C. for 30 s and by an extension step at 72° C. for 2 min. The PCR-amplified fragments were purified using the MinElute PCR Purification kit (reference LSKG ELO 50, Qiagen), according to the manufacturer's instructions.

The amplified fragments were digested at 37° C. for 1 h in a reaction mixture of 40 μl containing 2.5 IU of Bpm I (NEB) in a 50 mM Tris-HCL buffer, pH 7.9, 100 mM NaCl, 10 mM MgCl₂, 1 mM DTT and 100 μg/mL BSA.

The enzymes and the buffers were then eliminated by filtration (Microcon YM3, Millipore) and the DNA retained on the filter was eluted using the Micropure-EZ kit (Millipore), then the short fragments were purified by filtration (Microcon YM 30, Millipore); the DNA fragments of less than 100 bp corresponding to the eluate, the larger fragments being retained on the filter.

The short fragments were then digested at 37° C. for 1 h in a reaction volume of 40 μl containing 5 IU of alkaline phosphatase and 3 IU of 5′ exonuclease in a 500 mM Tris-HCl-1 mM EDTA buffer, pH 8.5, and the reaction was then stopped by heating at 90° C. for 3 min.

The single-stranded target DNA fragments labeled with a fluorophore thus obtained were conserved with a view to subsequent use for the hybridization with a nucleotide probe or nucleotide probes.

### Example 2

### Analysis of the Short Fragments Obtained by Digestion with a IIS-Type Restriction Enzyme According to the Method of the Invention

1) Preparation of Labeled Long Fragments Containing at One of their Ends the Recognition Site for a IIS-Type Restriction Enzyme

### a) PCR Amplification

Long fragments referred to as lf1, lfz, lf4 and lf5, having, respectively, the sequences SEQ ID NO: 4 to SEQ ID No: 7, were amplified by polymerase chain reaction (PCR) using the following pairs of primers:

If1
(SEQ ID NO: 8)
sense primer: 5′ CGATGAGTGCTGACCGA 3′
(SEQ ID NO: 9)
antisense primer: 5′ GTAGACTGCGATGCG 3′
If2, If 4 and If5
(SEQ ID NO: 10)
sense primer: 5′ CGATGAGTGCTGA 3′
(SEQ ID NO: 9)
antisense primer: 5′ GTAGACTGCGATGCG 3′.

The recognition site for the Bpm I restriction enzyme (5′CTGGAG3′) or Mme I restriction enzyme (5′ TCCPuAC3′) was introduced at the 5′ end of the products thus obtained, by means of a second PCR amplification using the following pair of primers:

Bpm I
(SEQ ID NO: 11)
sense primer: 5′ CGATGACTGGAGACCGA 3′
(SEQ ID NO: 9)
antisense primer: 5′ GTAGACTGCGATGCG 3′
Mme I
(SEQ ID NO: 12)
sense primer: 5′ CGATGAGTTCCGACCGA 3′
(SEQ ID NO: 9)
antisense primer: 5′ GTAGACTGCGATGCG 3′

### b) Labeling

The modified long fragments obtained in a) were labeled at their 5′ end, either with γ³²P-ATP or with a fluorophore, such as cyanine 3 (Cy3) or fam.

More specifically, the PCR products (2 μl) obtained in a) are denatured by heating to 80° C. and immediately transferred into liquid nitrogen, and then 1 μl of a labeling mixture containing polynucleotide kinase (PNK, 30 IU) and 2 μl of ATPγ³²P, in a final volume of 50 μl of buffer for this enzyme, are added and the labeling is carried out at 37° C. for 30 minutes. The radiolabeled products are then purified on a G25 exclusion column.

2) Analysis of the Radiolabeled Short Fragments Obtained after Cleavage with Bpm I
a) Cleavage with Bpm I

The radiolabeled PCR products purified as above are dissolved in Bpm I enzyme buffer (5×, 4 μl), and then hybridized again by heating at 80° C. followed by a slow return to ambient temperature; 16 μl of H₂O are then added and 4 μl of the final mixture (20 μl) are removed for digestion. The restriction enzyme is then added (2 units, i.e. 1 μl; New England Biolabs), along with 0.2 μl of bovine serum albumin (10 mg/ml) and 1 μl of enzyme buffer, in a final volume of 10 μl. Aliquot fractions of 2 μl are removed at various times (15, 30, 75 and 120 minutes) in order to follow the progress of the reaction. The reactions are stopped by adding 2 μA of a solution of formamide containing bromophenol blue and xylene cyanol and then heating the mixture at 80° C. for 3 minutes. The 2 μl of remaining cleavage product are digested as specified below.

b) Digestion with Alkaline Phosphate (AP) and 5′ Exonuclease (PDE II)

The remaining product from cleavage with Bpm I (2 μl) is then treated with alkaline phosphatase (p5521, Sigma, 1000 U/40 μl in 3.2M ammonium sulfate buffer, pH 7) for 15 minutes at 37° C., and then with PDE II (P9041, Sigma, 10⁻¹ U/μl in 2M ammonium citrate buffer, pH 5.5) for 30 minutes at 37° C. The digestion product thus obtained corresponds hereinafter to the final time T.

### c) Polyacrylamide Gel Analysis of the Cleavage Products Obtained

FIG. 5 represents the restriction map for the long fragments lf2 and lf4 with Bpm I. More specifically, the cleavage of lf2 with Bpm I generates the following fragments, i.e.: fragments of 28 and 131 base pairs (bp) by cleavage downstream of the recognition site for Bpm I located in the 5′ position, generated by PCR, fragments of 115 and 44 bp cleavage downstream of the second site for Bpm I (internal site present only in lf2), and fragments of 28, 85 and 44 bp cleavage downstream of the two recognition sites above. The cleavage of lf4 with Bpm I generates fragments of 28 and 23 nucleotides.

The polyacrylamide (20%) gel analysis of the kinetics of cleavage of the fragments lf2 and 114 with Bpm I (FIG. 6) shows the presence of fragments of approximately 131, 115, 85 and 45 bp for lf2 and of fragments of 23 and 28 bp for lf4, indicating that cleavage with the Bpm I enzyme is effective from 15 minutes onward. The disappearance of the signal at the final time T indicates that the digestion with alkaline phosphatase and the 5′ exonuclease PDE II is effective.

3) Analysis of the Short Fragments Labeled with a Flourophore, Obtained after Cleavage with Bpm I or Mme I

The short fragments labeled with a fluorophore, obtained after cleavage with Bpm I or Mme are analyzed using a bioanalyzer (Agilent), which comprises separation of the DNA by gel electrophoresis and detection of the various fragments by measuring the amount of fluorescence emitted by an intercalating agent specific for the double-stranded DNA; this technique does not make it possible to detect double-stranded DNA fragments of less than 25 bp in size and single-stranded DNA fragments.

### a) Protocol

The modified fragment lf2, labeled with a fluorophore (4 μl), prepared as above is incubated at 37° C. for 3 hours in a 10 μl reaction mixture containing 1 μl of buffer number 3 (10×; New England Biolabs), 4.4 μl of H₂O, 0.2 μl of bovine serum albumin and 0.5 μl of Bpm I (1 unit; New England BioLabs). Five microliters of the cleavage product are analyzed on the bioanalyzer and the remaining 5 μl are treated with 2 μl of alkaline phosphatase (P5521, Sigma, 1000 U/40 μl in 3.2M ammonium sulfate buffer, pH 7) for 15 minutes at 31° C., followed by incubation for 30 minutes at 37° C. with 0.5 μl of PDE II (P9041, Sigma, 10⁻¹ V/μl 2M ammonium citrate buffer, pH 5.5).

Alternatively, the modified fragment lf2, labeled with a fluorophore (4 μA, prepared as above is incubated for 3 hours at 37° C. in a 10 μl reaction mixture containing 1 μl of buffer number 4 (10×; New England Biolabs), 3.5 μl of H₂O, 1 μl of SAM (S-adenosyl-methionine) and 0.5 μl of Mme I (1 unit). Five microliters of this digestion product are analyzed on the bioanalyzer and the remaining 5 μl are treated with alkaline phosphatase and with PDE II as above.

In addition, molecular weight markers are added to the mixture before analysis using the bioanalyzer, so as to identify the size of the fragments generated after cleavage with the restriction enzymes.

### b) Results

The restriction map for the fragment lf2 with Bpm I and Mme I is given in FIG. 7.

FIGS. 8 and 9 illustrate the profile of the fragments obtained after cleavage, respectively with Bpm I and Mme I, of the long fragment lf2 5′ labeled with Cy3 (in A) or fam (in B).

By comparison with the control profile (in the absence of restriction enzyme; upper panel), the results show that the cleavage with Bpm I is total (FIGS. 8A and 8B; central panel), whereas the cleavage with Mme I is partial (FIGS. 9A and 9B; central panel). In addition, the profile of the fragments 5′-labeled with Cy3 (in A) or fam (in B), cleaved with Bpm I or Mme I and digested with alkaline phosphatase and the 5′ exonuclease shows a decrease in the signal (FIGS. 8A, 8B, 9A and 9B; lower panel), indicating that there is digestion of the DNA by these enzymes but that this digestion is only partial. However, this type of analysis, which is specific for the double-stranded DNA, does not make it possible to verify the total digestion of the DNA strand not 5′-coupled to the fluorophore, the protection of the DNA strand 5′-coupled to the fluorophore and the presence of the short single-stranded fragment 5′-labeled with a fluorophore that results therefrom.

### Example 3

### Use of the Target DNAs for Hybridizing Oligonucleotide Probes

### 1) Materials and Methods

A glass support of the DNA chip type (Codelink slides), on which are immobilized oligonucleotide probes, some of which are complementary to the target DNA fragments obtained in example 1 or 2, was prepared according to techniques known in themselves. Said target DNAs (1.5 μl) were then diluted in hybridization buffer (H1740, Sigma; 1.5 μl) and 10 μl were deposited onto the glass support, between slide and coverslip (round coverslip 12 mm in diameter). The hybridization was then carried out in a humid chamber in a thermocycler, under the following conditions: 80° C. for 3 min, then the temperature is decreased to 50° C. in steps of 0.1° C./s and, finally, the temperature is maintained at 50° C. for 10 minutes. The hybridization reaction is then stopped by depositing the glass slides on ice. Alternatively, the hybridization is carried out in a ventilated oven at 39° C. for 30 minutes.

The excess of target DNA fragments not complementary to the probes is then eliminated by means of successive washes: 1 wash for 20 s to 30 s with 2×SSC (Sigma, S6639), 1 wash for 20 s to 30 s with 2×SSC to which 0.1% SDS has been added (L4522, Sigma) and 3 washes for 20 s to 30 s with 0.2×SSC, at +4° C.

The glass slides were then dried and the hybridization was visualized and analyzed using a scanner (Genetac model, Genomic Solution).

### 2) Results

The hybridization of the following targets prepared as described in example 2 was compared:
short fragment lf2 5′-labeled with Cy3, generated by cleavage with Bpm I;
short fragment lf2 5′-labeled with Cy3, generated by cleavage with Bpm I, digested with alkaline phosphatase and the 5′ exonuclease PDE II;
fragment lf2 5′-labeled with Cy3, not cleaved, not digested (control).

The results of the comparative analysis (FIG. 10 and table I) show that the intensity of the hybridization signal for the long double-stranded target is two-fold less than that for the short double-stranded fragments. The hybridization intensity is further increased when the short double-stranded fragments are converted to single-stranded fragments.

TABLE I
Comparative analysis of the intensity of the
hybridization signal for the various targets

| | | | Bpm I + |
|---|---|---|---|
| | Nontreated | | PA + |
| Intensity | control | Bpm I | PDE II |
| I max (×10³) | 13 | 20 | 23 |
| I mean | 7300 | 13 500 | 14 300 |

As emerges from the above, the invention is in no way limited to those of its methods of implementation, execution and application which have just been described more explicitly; on the contrary, it encompasses all the variants thereof which may occur to those skilled in the art, without departing from the context or from the scope of the present invention.

What is claimed:

1. A method of preparing DNA fragments, which comprises at least the following steps of:
a) preparing double-stranded DNA fragments from a sample of nucleic acids to be analyzed,
b) ligating the ends of said DNA fragments to a double-stranded oligonucleotide adaptor (adaptor AA′) comprising the recognition site for a restriction enzyme, the cleavage site of which is located downstream of said recognition site,
c) amplifying the fragments linked to said adaptor, using a pair of suitable primers, at least one being optionally labeled at its 5′ end, and
d) cleaving said DNA fragments close to one of their ends, using said restriction enzyme, so as to generate short fragments.

2. The method as claimed in claim 1, wherein steps a) and b) are carried out simultaneously.

3. The method of preparing DNA fragments as claimed in claim 1, which comprises an additional step consisting in purifying the fragments of less than 1000 bp, prior to the ligation step b).

4. The method of preparing DNA fragments as claimed in claim 1, wherein said adaptor comprises, upstream of the recognition site (zone 2), a zone 3 of at least 6 bp.

5. The method of preparing DNA fragments as claimed in claim 1, wherein said adaptor comprises on one of the strands (A or A′), downstream of the recognition site (zone 2), a zone 1 complementary to the sequence of the ends of the double-stranded DNA fragments of step a).

6. The method of preparing DNA fragments as claimed in claim 5, wherein said adaptor comprises at least one base located between zone 1 and zone 2 that is different from that which, in said restriction site, is immediately adjacent to the complementary sequence corresponding to zone 1.

7. The method of preparing DNA fragments as claimed in claim 1, wherein said adaptor comprises a phosphate residue covalently linked to the 5′ end of the strand A′.

8. The method of preparing DNA fragments as claimed in claim 1, wherein one of the primers is linked at its 5′ end to a suitable label.

9. The method of preparing DNA fragments as claimed in claim 1, wherein said primers contain, at their 3′ end, several bases specific for an informative sequence or informative sequence to be detected.

10. The method of preparing DNA fragments as claimed in claim 1, wherein one of the strands of the product amplified in step c) is protected at its 5′ end with a suitable label.

11. The method of preparing DNA fragments as claimed in claim 1, which comprises an additional step e) consisting in obtaining, by any suitable means, single-stranded fragments from the short fragments obtained in step d).

12. The method of preparing DNA fragments as claimed in claim 1, which comprises an additional step e′), consisting in purifying the short fragments obtained in step d), or a step f) consisting in purifying the single-stranded fragments obtained in step e).

13. A single-stranded DNA fragment produced by the method of claim 1, which is less than 100 bases or base pairs long and in that it comprises at least one informative sequence bordered at its 5′ and 3′ ends, respectively, by the recognition site and the cleavage site for a restriction enzyme that cleaves at a distance from its recognition site.

14. The single-stranded DNA fragment as claimed in claim 13, which is labeled at its 5′ end with a suitable label.

15. A DNA chip, which comprises a single-stranded DNA fragment as claimed in claim 13.

16. A method of hybridizing nucleic acids, which comprises hybridizing nucleic acids with:
a) a probe or a target consisting of a short single-stranded DNA fragment as claimed in claim 13, or
b) a probe consisting of a short double-stranded DNA fragment formed from the association of the short single-stranded DNA fragment as claimed in claim 13 and of the sequence complementary to said fragment.

17. A kit for carrying out nucleic acid hybridization, which comprises at least one DNA fragment as defined in claim 16 and a nucleic acid molecule complementary to said fragment.

18. An adaptor, which is formed from a double-stranded oligonucleotide (AA′) of at least 10 bp comprising, from 5′ to 3′:
a) a zone 3 of at least 6 bp,
b) a zone 2 comprising the recognition site for a restriction enzyme, the cleavage site of which is located downstream of the recognition site,
c) a zone 1 complementary to the sequence as defined in claim 5,
d) at least one base locate between zone 1 and zone 2 that is different from that which, in said restriction site, is immediately adjacent to the complementary sequence of said zone 1, and
e) a phosphate residue covalently linked to the 5′ end of the strand A′.

19. A primer, having a sequence selected from the group consisting of: the sequence of the oligonucleotide A of the adaptor as defined in claim 1, and the sequence of the latter, to which are added, in the 3′ position, bases corresponding to the sequence as defined in claim 5.

20. A kit for preparing DNA fragments which comprises at least one adaptor as claimed in claim 20.

21. A kit for preparing DNA fragments, which comprises at least one primer as claimed in claim 21.

22. A DNA chip, which comprises a single-stranded DNA fragment as claimed in claim 14.

23. A method of hybridizing nucleic acids, which comprises hybridizing nucleic acids with:
a) a probe or a target consisting of a short single-stranded DNA fragment as claimed in claim 14, or
b) a probe consisting of a short double-stranded DNA fragment formed from the association of the short single-stranded DNA fragment as claimed in claim 14 and of the sequence complementary to said fragment, or both a) and b).

24. A kit for carrying out nucleic acid hybridization, which comprises at least one DNA fragment as defined in claim 27 and a nucleic acid molecule complementary to said fragment.

25. The method of preparing DNA fragments as claimed in claim 1, wherein steps a) and b) are carried out simultaneously; and said adaptor comprises upstream of the recognition site (zone 2), a zone 3 of at least 6 bp, and downstream of the recognition site (zone 2), a zone 1 complementary to the sequence of the ends of the double-stranded DNA fragments of step a).

26. The kit of claim 22, wherein said DNA fragments are prepared by a method comprising at least the following steps of:
a) preparing double-stranded DNA fragments from a sample of nucleic acids to be analyzed,
b) ligating the ends of said DNA fragments to a double-stranded oligonucleotide adaptor (adaptor AA′) comprising the recognition site for a restriction enzyme, the cleavage site of which is located downstream of said recognition site,
c) amplifying the fragments linked to said adaptor, using a pair of suitable primers, at least one being optionally labeled at its 5′ end, and
d) cleaving said DNA fragments close to one of their ends, using said restriction enzyme, so as to generate short fragments.

27. The kit of claim 24, wherein said DNA fragments are prepared by a method comprising at least the following steps of:
a) preparing double-stranded DNA fragments from a sample of nucleic acids to be analyzed,
b) ligating the ends of said DNA fragments to a double-stranded oligonucleotide adaptor (adaptor AA′) comprising the recognition site for a restriction enzyme, the cleavage site of which is located downstream of said recognition site,
c) amplifying the fragments linked to said adaptor, using a pair of suitable primers, at least one being optionally labeled at its 5′ end, and
d) cleaving said DNA fragments close to one of their ends, using said restriction enzyme, so as to generate short fragments.


SEQUENCE LISTING (21 sequences)

SEQ ID NO: 1 - DNA, 17 bp, Artificial Sequence
  Description of Artificial Sequence Synthetic oligonucleotide
ggaagcctagctggagc

SEQ ID NO: 2 - DNA, 21 bp, Artificial Sequence
  Description of Artificial Sequence Synthetic oligonucleotide
aattgctccagctaggcttcc

SEQ ID NO: 3 - DNA, 21 bp, Artificial Sequence
  Description of Artificial Sequence Synthetic primer
ggaagcctagctggagcaatt

SEQ ID NO: 4 - DNA, 128 bp, Artificial Sequence
  Description of Artificial Sequence Synthetic oligonucleotide
cgatgagtgctgaccgagctgtatttgggacctggggtcttttccgaacgatgcacggagaaactgccccttcgtgttgacttcattcacagggtggagttcggagaggtgtccgcatcgcagtctac

SEQ ID NO: 5 - DNA, 157 bp, Artificial Sequence
  Description of Artificial Sequence Synthetic oligonucleotide
cgatgagtgctgaccgagctgtatttggaacctggggttttttccgaacgatgcacggaaaaactgccccttcgtgttgactgcattcacaggctggagttcggagaggtgtccgggcatcgggttcttatcaagaggggaccgcatcgcagtctac

SEQ ID NO: 6 - DNA, 49 bp, Artificial Sequence
  Description of Artificial Sequence Synthetic oligonucleotide
cgatgagtgctgaccctctctccggcgaacagcccgcatcgcagtctac

SEQ ID NO: 7 - DNA, 89 bp, Artificial Sequence
  Description of Artificial Sequence Synthetic oligonucleotide
cgatgagtgctgaccgactctttgcaaccctgtgaatcccagcacgccaggcctccctgtccatcaccaactcccgcatcgcagtctac

SEQ ID NO: 8 - DNA, 17 bp, Artificial Sequence
  Description of Artificial Sequence Synthetic primer
cgatgagtgctgaccga

SEQ ID NO: 9 - DNA, 15 bp, Artificial Sequence
  Description of Artificial Sequence Synthetic primer
gtagactgcgatgcg

SEQ ID NO: 10 - DNA, 13 bp, Artificial Sequence
  Description of Artificial Sequence Synthetic primer
cgatgagtgctga

SEQ ID NO: 11 - DNA, 17 bp, Artificial Sequence
  Description of Artificial Sequence Synthetic primer
cgatgactggagaccga

SEQ ID NO: 12 - DNA, 17 bp, Artificial Sequence
  Description of Artificial Sequence Synthetic primer
cgatgagttccgaccga

SEQ ID NO: 13 - DNA, 137 bp, Artificial Sequence
  Description of Artificial Sequence Synthetic oligonucleotide
cgatgagtgctgctggagctgtatttnnnnnnnnnnnnacctggggtcttttccgaacgatgcacggagaaactgccccttcgtgttgacttcattcacagggtggagttcggagaggtgtccgcatcgcagtctac

SEQ ID NO: 14 - DNA, 34 bp, Artificial Sequence
  Description of Artificial Sequence Synthetic oligonucleotide
cgatgagtgctgctggagctgtatttggaacctg

SEQ ID NO: 15 - DNA, 46 bp, Artificial Sequence
  Description of Artificial Sequence Synthetic oligonucleotide
ggaagcctagctggagcaatttggaacctggggtcttttccgaacg

SEQ ID NO: 16 - DNA, 32 bp, Artificial Sequence
  Description of Artificial Sequence Synthetic oligonucleotide
ggaagcctagctggagcaatttggaacctggg

SEQ ID NO: 17 - DNA, 14 bp, Artificial Sequence
  Description of Artificial Sequence Synthetic oligonucleotide
gtcttttccgaacg

SEQ ID NO: 18 - DNA, 16 bp, Artificial Sequence
  Description of Artificial Sequence Synthetic oligonucleotide
cgttcggaaaagaccc

SEQ ID NO: 19 - DNA, 32 bp, Artificial Sequence; identical to SEQ ID NO: 16
  Description of Artificial Sequence Synthetic oligonucleotide

SEQ ID NO: 20 - DNA, 32 bp, Artificial Sequence; identical to SEQ ID NO: 16
  Description of Artificial Sequence Synthetic oligonucleotide

SEQ ID NO: 21 - DNA, 10 bp, Artificial Sequence
  Description of Artificial Sequence Synthetic oligonucleotide
ggaagcctag